(19) United States
(12) Patent Application Publication
(10) Pub. No.: US 2010/0217356 A1
(43) Pub. Date: Aug. 26, 2010

(54) Method to reduce heating at implantable medical devices including neuroprosthetic devices

(76) Inventors: Marom Bikson, New York, NY (US); Maged M. Elwassif, Long Island City, NY (US); Qingjun Kong, Elmhurst, NY (US)

Correspondence Address: Bernard S. Hoffman, 10 Colgate Street, Port Jefferson Station, NY, 11776, US

(21) Appl. No.: 12/380,021

(22) Filed: Feb. 21, 2009

(51) Int. Cl. A61N 1/375

(52) U.S. Cl. 607/63

(57) ABSTRACT

A method to control tissue/device heating at implantable medical devices including neuroprosthetic devices. In a first embodiment, thermal conductivity of components of the implantable medical devices including the neuroprosthetic devices is increased. In a second embodiment, the implantable medical devices including the neuroprosthetic devices are cooled by using heat-sinks. In a third embodiment, portions of the implantable medical devices including the neuroprosthetic devices are replaced with specific thermal properties. In a fourth embodiment, the implantable medical devices including the neuroprosthetic devices are coated with a drug/material that will induce surrounding tissue to become more resistant to temperature increases. In a fifth embodiment, the temperature increase near the implantable devices including the neuroprosthetic devices is determined using a modified bio-heat transfer model. In a sixth embodiment, the shape of the outer or the inner surface of the device is modified.

### THE CROSS REFERENCE TO RELATED APPLICATIONS

The instant nonprovisional patent application is a national patent application claiming priority from PCT international patent application number PCT/US2007/018484, filed on 21 Aug. 2007, and entitled METHOD TO REDUCE HEATING AT IMPLANTABLE MEDICAL DEVICES INCLUDING NEUROPROSTHETIC DEVICES, which claims priority from provisional patent application No. 60/839,002, filed on Aug. 21, 2006, and entitled METHOD TO REDUCE HEATING AT IMPLANTABLE MEDICAL DEVICES INCLUDING NEUROPROSTHETIC DEVICES, and which are both incorporated herein by reference thereto.

### THE BACKGROUND OF THE INVENTION

A. The Field of the Invention

The embodiments of the present invention relate to a method to reduce heating or to change spatial distribution of heating, and more particularly, but not by way of limitation, the embodiments of the present invention relate to a method to reduce heating or to change spatial distribution of heating at implantable medical devices including neuroprosthetic devices.

B. The Description of the Prior Art

Implantable medical devices are commonly used today to treat patients suffering from various ailments. Implantable medical devices, such as pacemakers, Deep Brain Stimulation (DBS), and glucose pumps, can cause heating of the device and surrounding tissue. The heating can result from:
Power consumption by the device—including internal batteries and external power delivery.
Device faults/failure.
Improper device use.
Electrical currents generated by the—normal—device operation—in the case of electrical stimulation devices/tissue Joule Heating.
Device coupling with an external electromagnetic field—for example MRI.
Device action(s) on tissue or interaction with another device.

These sources of heating may be cumulative and can result in poor device performance, unwanted effects on the tissue/device, and/or damage to the tissue/device. Tissue/device damage can lead to lasting morbidity and/or death. For all these reasons, it is important to control heating around implantable devices.

Medical devices that employ electrical stimulation in some aspect of their function can be referred to as neuroprosthetic devices. One example of a neuroprosthetic device is Deep Brain Stimulation (DBS).

DBS is a technology pioneered by Medtronic Corp, is FDA approved for the treatment of Parkinson's disease, and is under clinical trials for depression, epilepsy, and a range of other neurological disorders. DBS involves implantation of a lead inside the brain and electrical stimulation through this lead. As had been said, a side-effect of DBS is heating near the lead. For example, heating can result from:
Electrical stimulation during normal DBS lead operation.
Coupling of DBS leads with external magnetic fields, such as those generated during MRI or Diathermy treatment.

Excessive heating can lead to tissue ablation, brain damage, and death. Heating as a result of coupling with external magnetic fields has recently become a significant DBS safety concern. In particular:
Two DBS patients suffered severe brain damage after undergoing Diathermy treatment.
Computer modeling and ‘phantom’ experimental studies have indicated that MRI fields can result in tissue destruction in DBS patients.
Additional case studies on brain damage due to heating may be forthcoming.

As a result, Medtronic has altered counter-indication protocols and issued a voluntary recall. These measures have not completely ameliorated this critical safety problem because:
Significant unknowns remain about heating risks, i.e., are the new guideline sufficient, i.e., what additional—potentially lethal—counter-indications have not yet been identified.
Because MRI is an important tool in treating and evaluating DBS patients, restrictions on MRI use impair patient care and technology development.
Future advances in DBS technology targeting new diseases/patient populations may be hampered by these safety concerns. It is also noted that patients and clinicians are generally not confident in these new guidelines, e.g., Clinicians note unexplained scanner-to-scanner variability and DBS patients refuse to enter even approved MRI scanners. Medtronic's concern oven this issue is further evident by a series of recent patents attempting to deal with this issue.

Concerns about heating are not limited to only DBS, and heating near implantable medical devices is of broad and significant concern.

The electrical stimulation of tissues can lead to temperature rises as a result of both Joule heat and metabolic responses to stimulation.¹ Electrical stimulation-induced changes in temperature can profoundly affect tissue function. Moderate temperature increases are not necessarily necrotic. ¹Tungjitusolmun S., E. J. Woo, H. Cao. Finite Element Analyses of Uniform Current Density Electrodes for Radio-Frequency Cardiac Ablation. IEEE Trans. Biomedical Engr., 2000; vol. 47, No. 1: 32-40; Labonte S. Numerical Model for Radio-Frequency Ablation of the Endocardium and its Experimental Validation. IEEE Trans. Biomed. Eng. 1994; vol. 41, No. 2:108-115; Chang I. Finite Element Analysis of Hepatic Radio-Frequency Ablation Probes Using Temperature-Dependent Electrical Conductivity. Biomedical Engineering Online 2003, 2:12; LaManna J C, K A McCracken, M Patil, O J Prohaska. Brain Tissue Temperature: Activation-Induced Changes Determined with a New Multisensor Probe. Exp. Med. Biol. 1988; 222: 383-9; LaManna J C, K A McCracken, M Patil, O J Prohaska. Stimulus-Activated Changes in Brain Tissue Temperature in the Anesthetized Rat. Metab. Brain Dis. 1989; 4(4): 225-37.

Joule heat will be produced in any electrical field where electrical currents are circulating.² The magnitude and spatial distribution of the induced temperature changes are a function of tissue properties and the electrical stimulation parameters. Electrical stimulation has been used as a tool to analyze brain metabolism and related temperature rises.³ Numerical models of radio-frequency ablation probes show that a voltage greater than 10 V R.M.S. will increase temperature to 40° C. or more.⁴ These reports indicate that reducing stimulation intensity reduces peak temperature rises, but did not explicitly examine stimulation voltages sufficient to induce moderate (<2° C.) temperature changes. Electrical stimulation in rat brains with micro-electrodes (1-10 s, 0.5 pulse duration, 10-20 Hz; at stimulation intensities below those generating seizures) has been shown to increase brain temperature up to 0.1 -0.5° C., 1 mm from the stimulating electrodes.⁵ ²LaManna J C, K A McCracken, M Patil, O J Prohaska. Stimulus-Activated Changes in Brain Tissue Temperature in the Anesthetized Rat. Metab. Brain Dis. 1989; 4(4): 225-37.³LaManna J C, M Rosenthal, R Novack, D F Moffett, F F Jobsis. Temperature Coefficients for the Oxidative Metabolic Responses to Electrical Stimulation in Cerebral Cortex. J Neurochem. 1980; 34(1): 203-9; LaManna J C, K A McCracken, M Patil, O J Prohaska. Brain Tissue Temperature: Activation-Induced Changes Determined with a New Multisensor Probe. Exp. Med. Biol. 1988; 222: 383-9; LaManna J C, K A McCracken, M Patil, O J Prohaska. Stimulus-Activated Changes in Brain Tissue Temperature in the Anesthetized Rat. Metab. Brain Dis. 1989; 4(4): 225-37; Tasaki I, P M Byrne. Heat Production Associated with Synaptic Transmission in the Bullfrog Spinal Cord. Brain Res. 1987; 407(2): 386-9.⁴Labonte S. Numerical Model for Radio-Frequency Ablation of the Endocardium and its Experimental Validation. IEEE Trans. Biomed Eng. 1994; vol. 41, No. 2:108-115; Chang I. Finite Element Analysis of Hepatic Radio-frequency Ablation Probes Using Temperature-Dependent Electrical Conductivity. Biomedical Engineering Online 2003, 2:12.⁵LaManna J C, K A McCracken, M Patil, O J Prohaska. Brain Tissue Temperature: Activation-Induced Changes Determined with a New Multisensor Probe. Exp. Med. Biol. 1988; 222: 383-9; LaManna J C, K A McCracken, M Patil, O J Prohaska. Stimulus-Activated Changes in Brain Tissue Temperature in the Anesthetized Rat. Metab. Brain Dis. 1989; 4(4): 225-37.

Brain function is especially sensitive to changes in temperature. An increase in temperature by ˜1° C. can have profound effects on single neuron and neuronal network function.⁶ For most membrane channels, the temperature dependence of conductance is comparable to that of a diffusion-limited process, while the temperature dependence of channel gating and pump kinetics can exceed this value by more than an order of magnitude.⁷ ⁶Hoffmann H M, V E Dionne. Temperature Dependence of Ion Permeation at the Endplate Channel. J. Gen. Physiol 1983; 81(5): 687-703; Moser E, I Mathiesen, P Andersen. Association Between Brain Temperature and Dentate Field Potentials in Exploring and Swimming Rats. Science. 1993; 259 (5099):1324-6; Stiles J R, I V Kovyazina, E E Salpeter, Salpeter Mm. The Temperature Sensitivity of Miniature Endplate Currents Is Mostly Governed by Channel Gating: Evidence from Optimized Recordings and Monte Carlo Simulations. Biophys. J 1999; 77(2): 1177-87; Bennetts B, M L Roberts, A H Bretag, G Y Rychkov. Temperature Dependence of Human Muscle C1C-1 Chloride Channel. J. Physiol. 2001; 535(Pt 1): 83-93; Fujii S, H Sasaki, K Ito, K Kaneko, H Kato. Temperature Dependence of Synaptic Responses in Guinea Pig Hippocampal Cal Neurons In Vitro. Cell Mol. Neurobiol. 2002; 22(4): 379-91.⁷Bennetts B, M L Roberts, A H Bretag, G Y Rychkov. Temperature Dependence of Human Muscle C1C-1 Chloride Channel. J. Physiol. 2001; 535(Pt 1): 83-93; Dostrovsky, J. O., R. Levy, J. P. Wu, W. D. Hutchison, R. R. Tasker, A. M. Lozano. Microstimulation-Induced Inhibition of Neuronal Firing in Human Globus Pallidus. J. Neurophysiol 2000; 84: 570-574.

All biophysical properties, including those suggested to play a role in the effects of DBS, are temperature dependent. These include membrane properties, such as passive resistance/capacitance and voltage gated channel kinetics. Changes in membrane properties will affect firing threshold, peak firing rate,⁸ and depolarization block threshold in response to DBS.⁹ Excitatory and/or inhibitory synaptic transmission has been suggested to mediate the effects of DBS.¹⁰ Both are highly sensitive to temperature changes.¹¹ ⁸McIntyre C C, M Savasta, L Kerkerian, L Goff, J L Vitek. Uncovering the Mechanism(s) of Action of Deep Brain Stimulation: Activation, Inhibition, or Both. Chin. Neurophysiol. 2004b; 115(6): 1239-48.⁹Beunier C, B Bioulac, J Audin, C Hammond. High-Frequency Stimulation Produces a Transient Blockade of Voltage-Gated Currents in Subthalamic Neurons. J. Neurophysiol. 2001; 85(4): 1351-6.¹⁰Dostrovsky, J. O., R. Levy, J. P. Wu, W. D. Hutchison, R. R. Tasker, A. M. Lozano. Microstimulation-Induced Inhibition of Neuronal Firing in Human Globus Pallidus. J. Neurophysiol 2000; 84: 570-574.¹¹Pierau F R, M R Klee, F W. Klussmann. Effect of Temperature on Postsynaptic Potentials of Cat Spinal Motoneurones. Brain Res. 1976; 114(1): 21-34; Hoffmann H M, V E Dionne. Temperature Dependence of Ion Permeation at the Endplate Channel. J. Gen. Physiol. 1983; 81(5): 687-703; Stiles J R, I V Kovyazina, E E Salpeter, Salpeter M M. The Temperature Sensitivity of Miniature Endplate Currents is Mostly Governed by Channel Gating: Evidence from Optimized Recordings and Monte Carlo Simulations. Biophys. J. 1999; 77(2): 1177-87; Fujii S, H Sasaki, K Ito, K Kaneko, H Kato. Temperature Dependence of Synaptic Responses in Guinea Pig Hippocampal Cal Neurons In Vitro. Cell Mol. Neurobiol. 2002; 22(4): 379-91.

Temperature dependent changes in pump kinetics will effect the regulation of the neuronal environment including the accumulation of neurotransmitters and ions. Extracellular potassium accumulation, which is associated with high-frequency electrical stimulation and has been suggested to play a role in DBS,¹² is highly sensitive to temperature changes and related metabolic activity.¹³ Research on the mechanisms of DBS has also focused on associated neurotransmitter concentration changes¹⁴ whose release and clearance kinetics will both change with temperature. Potential DBS-induced changes in brain temperature are thus of broad interest in quantifying the mechanisms of DBS. ¹²Bikson M, J Lian, P J Hahn, W C Stacey, C Sciortino, D M Durand. Suppression of Epileptiform Activity by High Frequency Sinusoidal Fields in Rat Hippocampal Slices. J. Physiol. 2001; 531 (Pt 1): 181-91; Lian J, M Bikson, C Sciortino, W C Stacey, D M Durand. Local Suppression of Epileptiform Activity by Electrical Stimulation in Rat Liippocampus In Vitro. J. Physiol. 2003; 547(Pt 2): 427-34.¹³Lewis D V, W H Schuette. Temperature Dependence of Potassium Clearance in the Central Nervous System. Brain Res. 1975; 99(1): 175-8; Lothman E, J Lamanna, G Cordingley, M Rosenthal, G Somjen. Responses of Electrical Potential, Potassium Levels, and Oxidative Metabolic Activity of the Cerebral Neocortex of Cats. Brain Res. 1975; 88(1): 15-36.¹⁴Windels F, N Bruet, A Poupard, N Urbain, G Chouvet, C Feuerstein, M Savasta. Effects of High Frequency Stimulation of Subthalamic Nucleus on Extracellular Glutamate and OABA in Substantia Nigra and Globus Pallidus in the Normal Rat. Eur. J. Neurosci. 2000; 12(11): 4141-6; Bruet N, F Windels, A Bertrand, C Feuerstein, A Poupard, M Savasta. High Frequency Stimulation of the Subthalaniic Nucleus Increases the Extracellular Contents of Striatal Dopamine in Normal and Partially Dopaniinergic Denervated Rats. J. Neuropathol Exp. Neurol. 2001; 60(1): 15-24; Savasta, M., F. Windels, N. Bruet, A. Bertrand, A. Poupard. Neurochemical Modifications Induced by High-Frequency Stimulation of Subthalamic Nucleus in Rats. In: Nichoisson, L., Editor. The basal ganglia VII, Kluwer, 2002:581-590; Urbano, F. J., E. Leznik, R. R. Llinas. Cortical Activation Patterns Evoked by Afferent Axons Stimuli at Different Frequencies: an In Vitro Voltage-Sensitive Dye Imaging Study. Thalamus Rel. Syst. 2002; 1:371-378; Lee K H, S Y Chang, D W Roberts, U Kim. Neurotransmitter Release from High-Frequency Stimulation of the Subthalamic Nucleus. J. Neurosurg. 2004; 101(3): 511-7.

Besides Joule heat, DBS may further increase brain temperature through increasing neuronal activity and concomitant metabolic activity, e.g., ion/neurotransmitter pumps.¹⁵ Indeed, DBS is generally associated with a local increase in metabolic activity.¹⁶ Both tissue heating and increased metabolic activity may promote increased blood flow as is observed during DBS.¹⁷ ¹⁵LaManna J C, K A McCracken, M Patil, O J Prohaska. Stimulus-Activated Changes in Brain Tissue Temperature in the Anesthetized Rat. Metab. Brain Dis. 1989; 4(4): 225-37; Tasaki I, P M Byrne.

Heat Production Associated with Synaptic Transmission in the Bullfrog Spinal Cord. Brain Res. 1987; 407(2): 386-9; Abbott B C, J V Howarth, J M Ritchie. The Initial Heat Production Associated with the Nerve Impulse in Crustacean and Mammalian Non-Myelinated Nerve Fibres. J. Physiol. 1965; 178: 368-83. ¹⁶Rezai A R, A M Lozano, A P Crawley, M L Joy, K D Davis, C L Kwan, J O Dostrovsky, R R Tasker, D J Mikulis. Thalamic Stimulation and Functional Magnetic Resonance Imaging: Localization of Cortical and Subcortical Activation with Implanted Electrodes. Technical Note. J. Neurosurg. 1999; 90(3): 583-90; Zonenshayn, M., A. Y. Mogilner, A. R. Rezai. Neurostimulation and Functional Brain Imaging. Neurol. Res. 2000; 22: 318-325; McIntyre C C, M Savasta, L Kerkerian, L Goff, J L Vitek. Uncovering the Mechanism(s) of Action of Deep Brain Stimulation: Activation, Inhibition, or Both. Chin. Neurophysiol. 2004b; 115(6): 1239-48.¹⁷J. S. Perlmutter, J. W. Mink, A. J. Bastian, K. Zackowski, T. Hershy, E. Miyawaki, W. Koller, T. O. Videen. Blood Flow Responses to Deep Brain Stimulation of Thalamus. Neurology 2002; 58: 1388-1394.

The range of physiological—non-necrotic—temperature transients, for example in response to sensory stimuli, remains unclear. Changes up to 0.12 to 1 .4° C. have been reported.¹⁸ Moderate (<2° C.) changes in brain temperature, for example induced by DBS, may thus exert a profound effect on neuronal function without leading to cell damage. ¹⁸LaManna J C, K A McCracken, M Patil, O J Prohaska. Stimulus-Activated Changes in Brain Tissue Temperature in the Anesthetized Rat. Metab. Brain Dis. 1989; 4(4): 225-37; Hoffmann H M, V E Dionne. Temperature Dependence of Ion Permeation at the Endplate Channel. J. Gen. Physiol. 1983; 81(5): 687-703.

Current DBS safety guidelines are presumably based solely on charge delivery/electrochemical considerations. While waveforms with reversal of stimulation phase—charge balanced stimulation—are advantageous from an electrochemical safety stand-point,¹⁹ they may be disadvantageous from a temperature safety stand-point—R.M.S. considerations. While lead/electrode selection does not necessarily factor in electrochemical safety considerations, thermal interaction between electrodes indicates that temperature-based safety guidelines must consider electrode separation distance. Therefore, the design of DBS stimulation parameters to limit temperature rises should follow separate guidelines then those to limit charge delivery. ¹⁹Merrill D R, M Bikson, J G Jefferys. Electrical Stimulation of Excitable Tissue Design of Efficacious and Safe Protocols. J. Neurosci. Methods. 2005; 141(2): 171-98.

Numerous innovations for medical implantable devices and their operation for treating diseases have been provided in the prior art, which will be described below, and which are incorporated herein by reference thereto. These innovations teach brain stimulation and do not address or even mention tissue heating resulting from normal device operation or unexpected factors, e.g., MRI coupling, as do the embodiments of the present invention.

(a) The U.S. Pat. No. 5,782,798 to Rise.

The U.S. Pat. No. 5,782,798 issued to Rise on Jul. 21, 1998 in class 604 and subclass 500 teaches techniques using one or more drugs and/or electrical stimulation for treating an eating disorder by way of an implantable signal generator and electrode and/or an implantable pump and catheter. A catheter is surgically implanted in the brain to infuse the drugs and one or more electrodes may be surgically implanted in the brain to provide electrical stimulation.

(b) The U.S. Pat. No. 5,800,474 to Benabid et al.

The U.S. Pat. No. 5,800,474 issued to Benabid et al. on Sep. 1, 1998 in class 607 and subclass 45 teaches a method of preventing seizures as experienced by persons with Epilepsy. High frequency electrical stimulation pulses are supplied to the subthalamic nucleus, thereby blocking neural activity in the subthalamic nucleus and reducing excitatory input to the substantia nigra, which leads to a reduction in the occurrence of seizures.

Further, numerous innovations for alternative methods for reducing hazardous tissue heating resulting from external magnetic coupling to DBS have been provided in the prior art, which will be described below, and which are incorporated herein by reference thereto.

Generally, these innovations teach modifying the geometry of the DBS lead wires or changing the material properties of the leads, so that coupling with external fields, e.g., MRI, is reduced, i.e., methods to generate MRI-safe implantable devices/leads sometimes in specific reference to tissue heating. These methods are unproven, may offer only minimal benefit even if practical, and are inherently complex leading to questions of clinical feasibility. Further, these “coupling-reducing” innovations are completely ineffective in reducing temperature rises induced by normal device function, including electrical stimulation or device faults.

These innovations generally focus on neuro-electrical stimulation devices, e.g., DBS in a MRI scanner. They attempt to reduce coupling of the device with the MRI field by either changing the device material properties or the device geometry. They do not suggest methods for reducing temperature rises once they are induced as do the embodiments of the present invention. Rather, they focus on reducing coupling with the MRI and hence reducing initial temperature generation. None of these innovations have been demonstrated to work in a person with an implantable device. Potentially, some of these innovations have been evaluated using rudimentary computer simulations or an experimental phantom—a fluid in a container inserted into a scanner. Both the computer simulation used and the phantom experiments—if they are used—have serious limitations in their applicability to humans. These innovations do not in any way address temperature increases resulting from device power consumption or from electrical currents induced by the stimulation device itself. Moreover, these innovations would in no way mitigate these other temperature increases because they only minimize coupling with an external magnetic field.

### (c) The United States Patent Application Publication Number 2005/0015128 to Rezai et al.

The United States Patent Application Publication Number 2005/0015128 published to Rezai et al. on Jan. 20, 2005 in class 607 and subclass 115 teaches a device and method for retaining an excess portion of a lead implanted within or on a surface of a brain of a patient. The device includes a burr hole ring configured to be secured to a skull of the patient and a lead retainer extending from the burr hole ring. The lead retainer is configured to store at least a section of the excess portion of the lead.

### (d) The United States Patent Application Publication Number 2005/0182482 to Wang et al.

The United States Patent Application Publication Number 2005/0182482 published to Wang et al. on Aug. 18, 2005 in class 623 and subclass 1.15 teaches a medical device including a coating-inhibiting distortion of medical-resonance images taken of the device. When the device is exposed to radio-frequency electromagnetic radiation with a frequency of from 10 megahertz to about 200 megahertz, at least 90 percent of this radio frequency electromagnetic radiation penetrates to the lumen of the device. The concentration of the radio frequency electromagnetic radiation penetrating to the lumen of the device is substantially identical at different points within the interior. The coating includes magnetic material with an average particle size of less than about 40 nanometers.

### (e) The United States Patent Application Publication Number 2005/0222642 to Przybyszewski et al.

The United States Patent Application Publication Number 2005/0222642 published to Przybyszewski et al. on Oct. 6, 2005 in class 607 and subclass 48 teaches an implantable stimulation system including a stimulator for generating electrical stimulation and a conductive stimulation lead having a proximal end electrically coupled to the stimulator. At least a first component of the impedance looking into the stimulator is substantially matched to the impedance of the stimulation lead. At least one distal stimulation electrode is positioned proximate the distal end of the stimulation lead. 2005/0222642 published to Przybyszewski et al.

### (f) The United States Patent Application Publication Number 2005/0222647 to Wahlstrand et al.

The United States Patent Application Publication Number 2005/0222647 published to Wahlstrand et al. on Oct. 6, 2005 in class 607 and subclass 72 teaches a pulse stimulation system configured for implantation into a patient's body, including a pulse stimulator, a conductive stimulation lead having a proximal end electrically coupled to the pulse simulator and having a distal end, and an electrode assembly coupled to the distal end of the stimulation lead. The electrode assembly includes an electrode body having a therapy electrode thereon being electrically coupled to the stimulation lead for delivering therapy to the patient. A floating electrode is configured to contact the patient's body tissue and has a surface area substantially larger than that of the therapy electrode. A filter is coupled between the therapy electrode and the floating electrode for diverting RF energy toward the floating electrode and away from the therapy electrode.

### (g) The United States Patent Application Publication Number 2005/0222656 to Wahlstrand et al.

The United States Patent Application Publication Number 2005/0222656 published to Wahlstrand et al. on Oct. 6, 2005 in class 607 and subclass 116 teaches a medical lead for use in a pulse stimulation system of the type including a pulse generator for producing electrical stimulation therapy. The lead includes an elongate insulating body and at least one electrical conductor within the insulating body. The conductor has a proximal end configured to be electrically coupled to the pulse generator and has a DC resistance in the range of 375-2000 ohms. At least one distal electrode is coupled to the conductor.

### (h) The United States Patent Application Publication Number 2005/0222657 to Wahlstrand et al.

The United States Patent Application Publication Number 2005/0222657 published to Wahlstrand et al. on Oct. 6, 2005 in class 607 and subclass 116 teaches a stimulation lead configured to be implanted into a patient's body, including at least one distal stimulation electrode and at least one conductive filer electrically coupled to the distal stimulation electrode. A jacket is provided for housing the conductive filer and provides a path distributed along at least a portion of the length of the lead for conducting induced RF energy from the filer to the patient's body.

### (i) The United States Patent Application Publication Number 2005/0222658 to Hoegh et al.

The United States Patent Application Publication Number 2005/0222658 published to Hoegh et al. on Oct. 6, 2005 in class 607 and subclass 116 teaches a neurostimulation lead configured to be implanted into a patient's body and has at least one distal electrode. The lead includes at least one conductive filer electrically coupled to the distal electrode, a jacket for housing the conductive filer, and a shield surrounding at least a portion of the filer for reducing electromagnetic coupling to the filer.

### (j) The United States Patent Application Publication Number 2005/0222659to Olsen et al.

The United States Patent Application Publication Number 2005/0222659 published to Olsen et al. on Oct. 6, 2005 in class 607 and subclass 116 teaches a lead configured to be implanted into a patient's body, including a lead body and a conductive filer positioned within the lead body and having a distal portion. An electrode is electrically coupled to the lead body and includes a stimulation portion, a bobbin, and at least one coil of wire wound on the bobbin and electrically coupled between the stimulation portion and the distal end region to form an inductor between the distal end region and the stimulation portion.

Numerous innovations for fabrication methods for implantable devices in no way address methods to mitigate temperature increases after implantation as do the embodiments of the present invention. Heat application or heat-sinks may be used in the fabrication process and are clearly not relevant to the embodiments of the present invention. For example:

### (k) The United States Patent Application Publication Number 2004/0215300 to Verness.

The United States Patent Application Publication Number 2004/0215300 published to Verness on Oct. 28, 2004 in class 607 and subclass 116 teaches conductive aerogels employed in fabrication of electrical medical leads adapted to be implanted in the body and subjected to bending stresses. An elongated, flexible, and resilient lead body extends from a proximal end to a distal end and includes an insulative sheath having an elongated lumen through which an elongated conductor extends. A layer of conductive aerogel is disposed over the conductor deforming upon movement of the conductor within the lumen against the aerogel in response to applied stresses.

Existing innovations using heat-sinks do not deal with neuron-prosthetic devices as do the embodiments of the present invention. For example, tissue ablation catheters are devices that are not chronically implanted. They are not implantable medical devices. Tissue ablation catheters deliberately induce tissue temperature increases for the purpose of destroying tissue. For example:

### (l) The United States Patent Application Publication Number 2003/0028185 to He.

The United States Patent Application Publication Number 2003/0028185 published to He on Feb. 6, 2003 in class 606 and subclass 41 teaches a self-cooling electrode for use with an ablation catheter having greater surface area, thereby allowing the electrode to dissipate heat to the blood pool more effectively and increased thermal mass and therefore greater heating capacity/thermal conductivity for improved heat transfer between the electrode and tissue for more effective tissue heating. The electrode design allows increased power to be delivered with minimized risk of overheating or coagulation at the tissue-electrode interface. The increased thermal mass and thermal conductivity of the electrode design are achieved with a substantially solid electrode body with thick walls. Cooling and increased heat exchange are achieved with an alternating pattern of channels and projections collectively defining a plurality of edges, either parallel or perpendicular to the electrode axis. Blood or other biological fluids can flow through the channels along the exterior surface of the electrode to help cool the electrode, while heat is simultaneously transferred from the electrode body, edges, and projections to the surrounding tissue. A catheter having a self cooled tip electrode in conjunction with one or more ring electrodes may be used to form a large virtual electrode capable of creating longer, deeper tissue lesions.

Although, He may deal with using a heat-sink, the heat-sink is used only during acute ablative electrical stimulation via a catheter, which is removed after tissue destruction, and is not an implanted device or a neuroprosthetic device. He suggests methods for controlling the spatial extent of tissue heating for ablation catheters including changing material properties.

Certain devices including implantable devices are designed to cool the body below normal level for therapeutic purposes and do not mitigate unwanted temperature increases generated by an implantable device that is not designed to change body temperature as do the embodiments of the present invention. For example:

### (m) The United States Patent Application Publication Number 2005/01 71585 to Saadat.

The United States Patent Application Publication Number 2005/0171585 published to Saadat on Aug. 4, 2005 in class 607 and subclass 96 teaches apparatus and methods for cooling selected regions within a body. An implantable cooling system is used to cool regions of the brain, spinal cord, fibrous nerve bodies, e.g., vagus nerve, etc. down to about 30° C. to diminish nerve impulses controlling seizures or chronic pain. The system includes an implantable unit containing a pumping mechanism and/or various control electronics. It also has a heat exchanger attachable to a tubular body organ, such as the superior vena cava or the inferior vena cava, through which the heat is effectively dissipated. Also included is a heat pump, such as a Peltier junction configured to be placed into contact with the region of tissue to be cooled. The heated portion of the Peltier junction is cooled by a liquid heat transfer medium absorbing the heat from the junction and dissipating it into the tubular body organ.

Electrical stimulation may be applied using non-implantable devices, for example, transcutaneous electrical stimulation, which are not implantable medical devices. For example:

### (n) The United States Patent Application Publication Number 2004/0204625 to Riehl.

The United States Patent Application Publication Number 2004/0204625 published to Riehl on Oct. 14, 2004 in class 600 and subclass 9 teaches a method for reducing discomfort caused by transcutaneous stimulation. The method includes providing transcutaneous stimulation, reducing the transcutaneous stimulation at a first location, and substantially maintaining the transcutaneous stimulation at a second location. The transcutaneous stimulation may be created by electric and/or magnetic fields. The first location may be relatively proximate to the cutaneous surface and may include tissue, nerves, and muscle. Also, the second location may be relatively deeper than the first location and include, for example, brain tissue requiring the transcutaneous stimulation for treatment purposes. The method further may include locating a conductor on a treatment area and/or a transcutaneous stimulation device relative to the first location. In addition, the method may further include adjusting how much the transcutaneous stimulation is reduced at the first location.

### (o) The United States Patent Application Publication Number 2005/0033382 to Single.

The United States Patent Application Publication Number 2005/0033382 published to Single on Feb. 10, 2005 in class 607 and subclass 57 teaches a device including a housing, electronic components contained within the housing, and a heat absorption medium sealed within the housing for regulating the temperature of the device. The heat absorption medium undergoes a state change at a state change temperature of 36° C. or greater. The device is a medical implant.

Single focuses on using a heat-absorption medium in contrast to transferring heat along/away from the device as do the embodiments of the present invention. In one embodiment, Single suggests using a heat-sink to channel heat generated in one region of the device to another region containing the heat-absorption material in contrast to not incorporating any heat-absorption material but rather spatially dissipating the heat over a wider region and then the heat is carried away by the tissue temperature regulation mechanism, e.g., blood flow as do the embodiments of the present invention.

Certain innovations deal with heat-absorption techniques. For example:

### (p) The United States Patent Application Publication Number 2005/0029990 to Tsukamoto et al.

The United States Patent Application Publication Number 2005/0029990 published to Tsukamoto et al on Feb. 10, 2005 in class 320 and subclass 135 teaches a method, device, and system for rapidly and safely discharging remaining energy stored in an electrochemical battery in the event of an internal short circuit or other fault. In its best mode of implementation, if a sensor detects one or more parameters, such as battery temperature or pressure exceeding a predetermined threshold value, the terminals of the battery or cell are intentionally short-circuited external to the battery through a low or near zero resistance load that rapidly drains energy from the battery. Heat generated by this rapid drain is absorbed by a heat absorbing material, such as an endothermic phase-change material like paraffin. The rate energy is drained via the external discharge load may be controlled with an electronic circuit responsive to factors, such as state of charge and battery temperature. Devices, such as inductive charging coils, piezoelectric and Peltier devices, may also be used as emergency energy discharge loads. Heat absorption material may be used to protect adjacent tissue in medically-implanted devices.

Thus, there exists a need for a “heat-sink” technology that dramatically reduces tissue/device heating, is inherently simple, and is evidently practical.

To mitigate these temperature changes, it is proposed by the embodiments of the present invention to integrate either into the device structure, inside the device—in the case of a hollow compartment, or outside the device, a passive heat-sink material or active heat-sink technology. For example, passive material with high thermal-conductivity that will act as a heat-sink and thus dissipate any temperature increases. Another example, different implementations of active heat-sink technologies may be used including those that incorporate fluid flow. In an additional implementation of the heat-sink technology, the device may be modified—for example coated with a drug—that will induce the surrounding tissue to become more resistant to temperature increases—for example by increasing tissue vasculature or changing of tissue properties. In specific cases, existing devices and already implanted devices may be modified or retrofitted based on this heat-sink technology. This heat-sink technology would be effective during ‘normal’ device operation and during unexpected/faulty operation. In an additional implementation, the heat generated near a device can be determined and used to guide device design including for neuroprosthetic devices electrical stimulation protocols.

### THE SUMMARY OF THE INVENTION

Thus, an object of the present invention is to provide a method to reduce heating or to change spatial distribution of heating at implantable medical devices including neuroprosthetic devices that avoids the disadvantages of the prior art.

Briefly stated, another object of the present invention is to provide a method to control tissue/device heating at implantable medical devices including neuroprosthetic devices. In a first embodiment, thermal conductivity of components of the implantable medical devices including the neuroprosthetic devices is increased. In a second embodiment, the implantable medical devices including the neuroprosthetic devices are cooled by using heat-sinks. In a third embodiment, portions of the implantable medical devices including the neuroprosthetic devices are replaced with specific thermal properties. In a fourth embodiment, the implantable medical devices including the neuroprosthetic devices are coated with a drug/material that will induce surrounding tissue to become more resistant to temperature increases. In a fifth embodiment, the temperature increase near the implantable devices including the neuroprosthetic devices is determined using a modified bio-heat transfer model. In a sixth embodiment, the shape of the outer or the inner surface of the device is modified.

The novel features which are considered characteristic of the present invention are set forth in the appended claims. The invention itself, however, both as to its construction and its method of operation and together with additional objects and advantages thereof will be best understood from the following description of the specific embodiments when read and understood in connection with the accompanying drawing.

### THE BRIEF DESCRIPTION OF THE DRAWING

The figures of the drawing are briefly described as follows:

FIG. 1 is a diagrammatic perspective view of the geometrical configuration of the model, wherein the brain tissue was modeled as a cylinder with a 5 cm radius and a 14 cm height, wherein the bottom—distal end—of the DBS lead was positioned in the center of the tissue (center), wherein two types of DBS leads were modeled including the 3389 DBS lead with 1.5 mm electrodes and 0.5 spacing between electrodes (right) and the 3387 lead with 1.5 mm electrodes and 1.5 mm spacing (left), and wherein the electrode index used is indicated;

FIG. 2 are maps of the bio-heat transfer model of DBS showing the effects of lead and electrode selection, wherein the false color maps indicate the spatial temperature distribution around the bipolar stimulating electrodes when the high stimulation setting was applied in a homogenous brain with tissue electrical conductivity σ=0.35 S/m, tissue thermal conductivity kₜ=0.527 W/m°, and no blood perfusion, wherein the red ‘axial’ line is the cross section at the proximal end of the most distal electrode—at height z=3 mm—extending in the r direction from the electrode, wherein in the remaining figures the temperature profile is plotted along this line, and wherein:

FIG. 2A is a map of lead 3387, wherein first and fourth electrodes were electrically energized;

FIG. 2B is a map of lead 3387, wherein first and second electrodes where electrically energized;

FIG. 2C is a map of lead 3389, wherein first and fourth electrodes where electrically energized; and

FIG. 2D is a map of lead 3389, wherein first and second electrodes are electrically energized;

FIG. 3 are graphs of temperature distribution along the axial direction when the high stimulation setting—lead 3389, electrodes 1 and 2—was applied in a homogenous brain, wherein:

FIG. 3A is a graph of temperature verses electrical conductivity (σ), wherein the thermal conductivity was fixed at 0.527 W/m° C. and blood perfusion was absent, and wherein tissue temperature increased with increasing electrical conductivity;

FIG. 3B is a graph of temperature verses thermal conductivity, wherein the electrical conductivity was fixed at 0.3 S/m and blood perfusion was absent, and wherein tissue temperature decreased with increasing thermal conductivity (Kₜ); and

FIG. 3C is a graph of temperature distribution verses blood perfusion, wherein electrical conductivity and thermal conductivity were constant—0.30 S/m and 0.527 W/m° C., respectively, and wherein blood temperature was 37° C. and metabolic heat was absent;

FIG. 4 are temperature distributions in a non-homogenous medium along the axial direction when the high stimulation setting—electrodes 1 and 2—was applied, wherein metabolic heat and blood perfusion were absent, and wherein:

FIG. 4A is a graph of temperature verses lead insulation thermal conductivity, wherein increasing the lead insulation thermal conductivity decreased the temperature around the electrode; and

FIG. 4B are maps of the temperature filed in the 3389 DBS lead and surrounding brain tissue with the lead insulation thermal conductivity equal to 0.026 W/m° C. (right) and equal to 20 W/m° C. (left), wherein a false color map indicates the spatial temperature distribution around the electrodes, and wherein the red line is the ‘axial’ cross section represented in other figures;

FIG. 5A is a graph showing the temperature distribution for normal thermal conductivity;

FIG. 5B is a map of the cross section of the electrode of normal thermal conductivity in the center of its conductive part;

FIG. 6A is a graph showing the temperature distribution after increasing the thermal conductivity;

FIG. 6B is a map of the cross section of the electrode of increased thermal conductivity in the center of its conductive part;

TABLE 1 illustrates the effects of biological parameters on peak temperature induced by DBS—high-setting, electrodes 1 and 2 were energized; and

TABLE 2 illustrates peak temperature verses insulation lead thermal conductivity (Kᵢ) at setting σ=0.3 S/m, Kₜ=0.527 W/m.K with no blood perfusion and metabolic heat, wherein high stimulation setting was applied to various combinations of leads and energized electrodes.

### THE DETAILED DESCRIPTION OF THE PREFERRED EMBODIMENTS

### A. General

Finite-element models are used to investigate how biological properties and DBS stimulation parameters affect the magnitude and spatial distribution of the DBS-induced temperature field. By solving the coupled Laplace equation of electrical field and the Pennes bioheat transfer equation, simulation of how DBS affects the temperature field distribution in brain tissue are enabled.

### B. The Model Methods

### (a) The General Description.

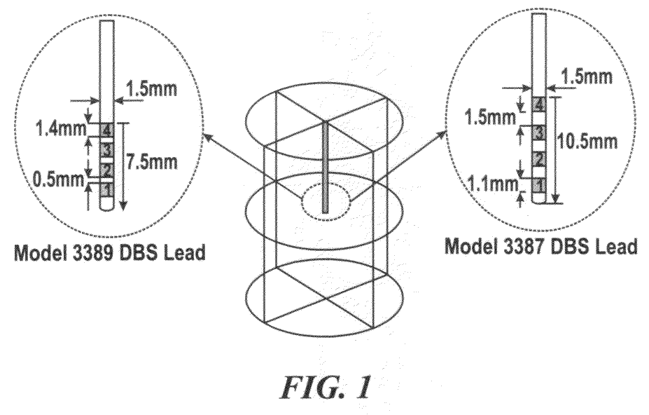

A bio-heat transfer model for DBS was developed implementing the Pennes model. Two types of Medtronic leads were studied, as shown in FIG. 1, the Model 3387 DBS lead with 1.5 mm spacing between each of the four electrodes at their distal ends to provide an electrodes spread of over a total of 10.5 mm and the Model 3389 DBS lead with 0.5 mm spacing between each of the four electrodes to provide an electrode spread of over a total of 7.5 mm. (Medtronic, Inc., Minneapolis, Minn.). Direct matrix inversion with 20,296 elements was used and doubling the resolution modulated temperature changes by <0.05° C.

Finite element analysis was used to analyze the effect of DBS on temperature rises in brain tissue. Because of axial geometrical symmetry, the temperature and electrical fields were assumed to vary only with direction of r—the coordinate of radius of the stimulator, and z—the coordinate of length of the stimulator. The three dimensional transfer model geometry can thus be mathematically implemented in two dimensions.

DBS-induced temperature increases in three increasingly detailed cases were examined:

### (a) The Case 1.

Temperature distribution induced by DBS in a homogenous brain tissue without blood perfusion or metabolic activity.

### (b) The Case 2.

Temperature distribution induced by DBS in a homogenous brain tissue with blood perfusion—solved using two dimensional Pennes model—with and without related metabolic activity.

### (c) The Case 3.

Temperature distribution induced by DBS in a non-homogenous brain tissue with consideration on physical properties of the DBS leads and tissue damage around the electrode.

In each of the above cases, the range of temperature increases expected in vivo by parameter sensitivity analysis was examined.

### (b) The Methods and Analysis.

Blood perfusion occurs in living tissues, and the passage of blood modifies the heat transfer in tissues. Furthermore, metabolic activity generates heat within the tissue. Pennes (1948) and Perl (1962) have established a simplified bio-heat transfer model to describe heat transfer in tissue by considering the effects of blood perfusion and metabolism.²⁰ During DBS, Joule heating arises when energy dissipated by an electric current flowing through a conductor is converted into thermal energy. The resulting bioheat equation—see EQUATION 1 below—governs heating during electrical stimulation.²¹ ²⁰F. A. Duck. Physical Properties of Tissues: A Comprehensive Reference Book, Academic Press, San Diego, 1990.²¹Tungjitusolmun S., E. J. Woo, H. Cao. Finite Element Analyses of Uniform Current Density Electrodes for Radio-Frequency Cardiac Ablation. IEEE Trans. Biomedical Engr., 2000; vol. 47, No. 1: 32-40; Chang I. Finite Element Analysis of Hepatic Radiofrequency Ablation Probes Using Temperature-Dependent Electrical Conductivity. Biomedical Engineering Online 2003, 2:12; Tungjitusolmun S., S. Tyler Staelin, D Haenmerich, J Z Tsai, H Cao, J G. Webster, V R. Vorperian. Three-Dimensional Finite-Element Analyses for Radio-Frequency Hepatic Tumor Ablation. IEEE Trans. Biomedical Engineering vol. 49, No. 1, January 2002.

ρ Cₚ δT/δt=∇(k ∇T)−ρ_{b} ω_{b} C_{b}(T−T_{b})+Qₘ+σ|∇V|² (1)

where:
ρ_{b} is the blood density (kg/m³)
C_{b} is the specific heat of the blood (J/kg.° C.)
k is the thermal conductivity of the brain tissue (W/m.° C.)
T is the temperature (° C.)
ω_{b} is the blood perfusion (ml/s/ml)
ρ is the brain tissue density
T_{b} is the body core temperature (° C.)
Qₘ is the metabolic heat (W/m³).

The Joule heat induced by DBS stimulation was modeled with a source term σ |∇V|², where σ is the electrical conductivity of the tissue and V is the electrical potential induced by stimulation.

The electrical potential was determined by solving the Laplace equation ∇.(σ∇V)=0.

Two normal DBS electrical settings were analyzed, but are not a limited set, ‘high setting’ (10 V, 185 pps, 210 μSec) with Vᵣₘₛ of 1.561 Volt, and ‘typical setting’ (3 V, 185 pps, 90 μSec) with Vᵣₘₛ of 0.353 Volt.²² ²²Implant Manual. Medtronic 3387, 3389 lead kit for Deep Brain Stimulation (2003).

The following range of tissue and lead parameters were applied, but are not a limited set:

(a) The Biological Properties of the Brain Tissue.²³ ²³Chang I. Finite Element Analysis of Hepatic Radiofrequency Ablation Probes Using Temperature-Dependent Electrical Conductivity. Biomedical Engineering Online 2003, 2:12; Baysal U, J Haueisen. Use of a Priori Information in Estimating Tissue Resistivities-Application to Human Data In Vivo. Physiol. Meas. 2004; 25: 737-748; Collins C, M Smith, R Tumer. Model of Local Temperature Changes in Brain upon Functional Activation. J. Appl. Physiol., 2004; 97(6): 2051-2055.
Kₜ Thermal conductivity of brain tissue (W/m.° C.)=0.5-0.6
ρ Density of brain tissue (kg/m³)=1040,
Cₚ Specific heat of brain tissue (J/kg.° C.)=3650
σ Electrical conductivity (S/m)=0.15-0.35
Tᵢ Initial Temperature of brain tissue=3 7° C.
   (b) The Biological Properties of the Blood.²⁴ ²⁴Collins C, M Smith, R Tumer. Model of Local Temperature Changes in Brain upon Functional Activation. J. Appl. Physiol., 2004; 97(6): 2051-2055; Xiaojiang X, P Tikuisis, G Giesbrecht. A Mathematical Model for Human Brain Cooling During Cold-Water Near-Drowning. Journal of Applied Physiology 86:265-272, 1999.
ω_{b} Volumetric blood perfusion rate per unit volume (ml/s/ml)=0.004-0.012,
ρ_{b} Density of blood (kg/m³)=1057
C_{b} Specific heat of blood (J/kg.° C.)=3 600,
T_{b} Body core temperature=36.7° C.

### (c) The Physical Properties of the DBS Lead Materials.

For the insulation portion of the leads (80A Urethane):²⁵ ²⁵Cengel, A Yunus, Turner, H Robert. Fundamentals of Thermal-Fluid Sciences, McGraw-Hill Science Pub Date: Mar. 30, 2004; Wei F. X, W. M. Grill. Current Density Distributions, Field Distributions and Impedance Analysis of Segmented Deep Brain Stimulation Electrodes. J. Neural Eng. 2 (2005) 139-147; David R. Lide, CRC Handbook of Chemistry and Physics. 81^{st} edition 2001.
Kᵢ (W/m.° C.)=0.026, ρᵢ (kg/m³)=1110, Cᵢ (J/kg.° C.)=1500, σᵢ (S/m)=10⁻¹⁰

For the electrode portion of the leads (Platinum/Iridium Pt 90/Ir 10):²⁶ ²⁶Goodfellow Corporation (DEVON, PA), Material Properties. http://www.goodfellow.com/csp/active/gfflome.csp; Wei F. X. and W. M. Grill. Current Density Distributions, Field Distributions and Impedance Analysis of Segmented Deep Brain Stimulation Electrodes. J. Neural Eng. 2 (2005) 139-147; David R. Lide, CRC Handbook of Chemistry and Physics. 81^{st} edition 2001.
Kₑ (W/m.° C.)=31, ρₑ (kg/m³)=21560, Cₑ (J/kg.° C.)=134, σₑ (S/m)=4*10⁶

### (d) The Dimensions and Boundary Conditions.

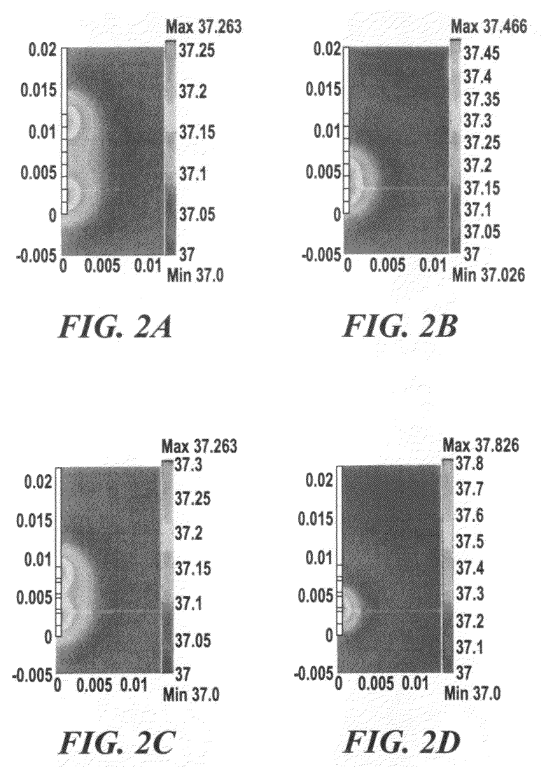

In order to obtain the particular solutions to the coupling temperature and electrical field, boundary conditions and initial conditions were required. The dimensions of the brain tissue that was modeled must be chosen appropriately to be large enough to abate boundary effects on temperature and electrical distribution close to the lead surface, as well as small enough to allow a reasonable computational time. In the model, the geometry of the brain tissue was set as a cylinder with a radius of 50 mm and a height of 140 mm, as shown in FIG. 1. For the boundary conditions of the electrical field, the voltage between the two energized electrodes, either 1 and 4, as shown in FIGS. 2A and 2C, or 1 and 2, as shown in FIGS. 2B and 2D, was set at Vᵣₘₛ. The outer boundaries of the brain tissue were treated as electrically insulated, namely δV/δn=0. For the thermal boundary conditions, the temperature at the outer boundaries of the brain tissue was fixed at 37° C., but the thermal boundary at the electrodes was set according to each case.

### (c) The Results.

(a) The Case 1—Temperature Distribution Induced by DBS in a Homogenous Brain Tissue without Blood Perfusion or Metabolic Activity.

With Case 1, how the temperature increases solely in response to DBS-induced Joule heat was focused on, without modeling the contribution of blood perfusion or metabolic activity. Therefore, both ω_{b} and Qₘ are zero. This model also treated the DBS electrode shaft to be electrically and thermally insulated, except that the electrodes 1 and 2—the two electrodes most distal on the DBS lead—were electrically energized.

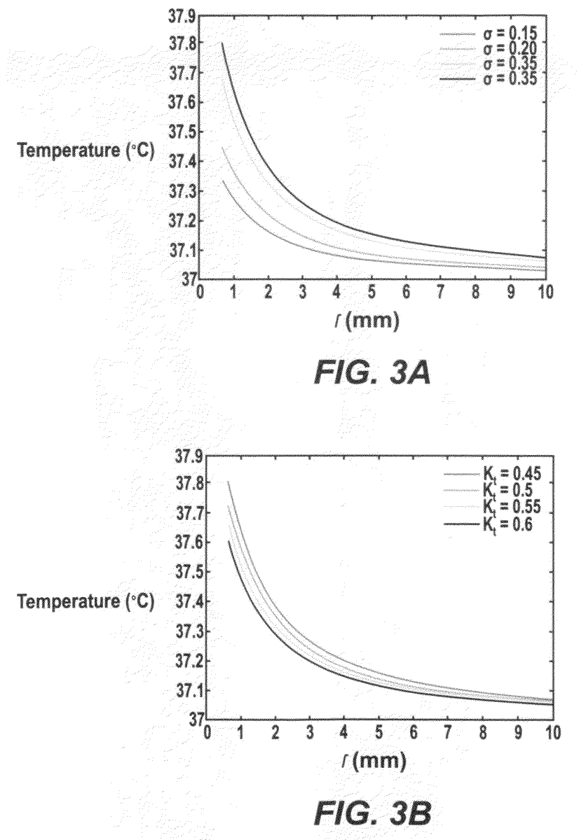

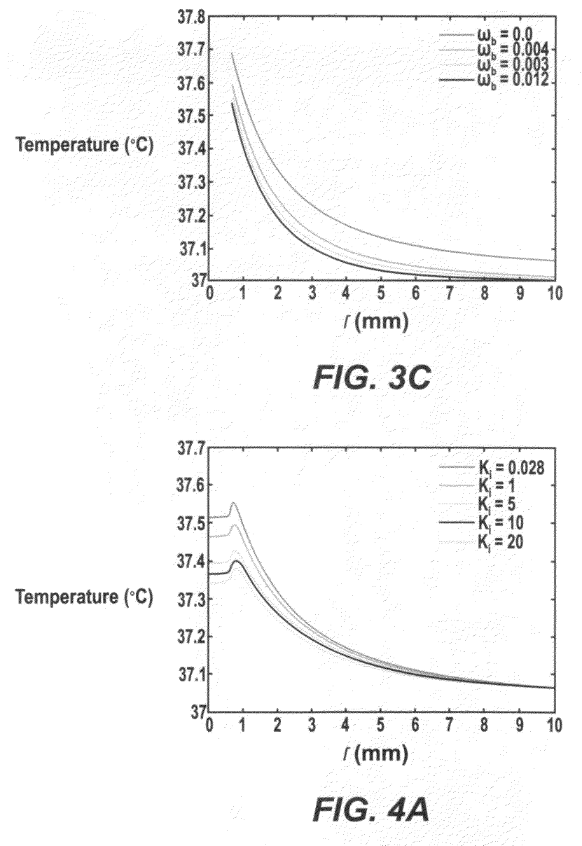

The temperature distributions using two types of DBS leads—Medtronic DBS Lead 3387 and Lead 3389—were modeled. ‘High setting’ to the two DBS leads was applied and how electrical conductivity and thermal conductivity affected the resulting temperature distribution in the brain tissue was investigated. FIG. 3A and TABLE 1, SECTION I show changes in peak temperature and temperature field distribution as a function of tissue electrical conductivity (σ=0.15 to 0.35 S/m) with thermal conductivity (Kₜ) fixed at 0.527 W/m.° C. FIG. 3B and TABLE 1, SECTION II show changes in peak temperature and temperature field distribution as a function of tissue thermal conductivity (0.45 to 0.60 W/m.° C.), with tissue electrical conductivity fixed at 0.3 S/m. The results show that temperature increases with electrical conductivity, while temperature decreases as thermal conductivity increases. Peak temperature on the lead's surface increased by 0.48° C. at σ=0.45 S/m and Kₜ=0.30 for Lead 3387 and 0.82° C. for Lead 3389, as shown in TABLE 1, SECTION I. The temperature field distribution using Lead 3387 was similar to that using Lead 3389. The temperature-field-space constant, defined here as the radial distance from the electrode that the temperature field decreased to 75%, as shown as the first contour line of FIG. 2, of its peak value—at the electrode surface—was not affected by changing in homogenous tissue electrical or thermal conductivity.

Across tissue parameters, the peak temperature for Lead 3389 was approximately 0.3° C. higher than that for Lead 3387, as shown in TABLE 1, SECTIONS I and II. This difference can be attributed to the increased distance between Lead 3387 electrodes, as shown in FIG. 2. For either Lead 3389 or Lead 3387, changing lead selection so that the leads where farther apart—e.g. leads 1 and 4—significantly reduced peak temperature increase, as shown in TABLE 2.

(b) The Case 2A—Temperature Distribution Induced by DBS in a Homogenous Brain Tissue with Blood Perfusion and No Metabolic Activity.

To study how the convection of blood regulates brain temperature during DBS, the blood perfusion rate, ω_{b}, was varied in the model from 0 to 0.012 ml/s/ml. In order to isolate how blood perfusion affected the temperature distribution, metabolic activity was not considered in this case of the model and blood temperature was fixed at 37° C. In this case—without metabolic activity—, the electrical conductivity and the thermal conductivity were fixed at 0.30 S/m and 0.527 W/m° C., respectively, and only the high-setting on DBS electrodes 1 and 2 was evaluated. As shown above, the temperature increased to 37.42° C. and 37.7° C. with leads model 3387 and 3389 under these conditions without blood perfusion, as shown in TABLE 1, SECTION I. The addition of blood perfusion convected Joule heat out of brain tissue so that the peak temperature decreased with increased blood perfusion, as shown in FIG. 3C. The peak temperature decreased moderately by 0.07° C. and 0.12° C. for Lead 3387 when the blood perfusion rates were 0.004 ml/s/ml and 0.012 ml/s/ml, respectively, as shown in TABLE 1, SECTION III. Similarly, decreases by 0.09° C. and 0. 16° C., respectively, occurred in the case using Lead 3389 when the blood perfusion rates were 0.004 ml/s/ml and 0.012 ml/s/ml, respectively. In contrast to the effects of changing tissue electrical/thermal conductivity, changes in blood perfusion rate effected brain temperature space constant. Increasing perfusion rate decreased the space constant—i.e., the temperature decreased over distance as a faster rate.

### (c) The Case 2B—Effects of Blood Perfusion and Metabolic Heat on Temperature Distribution Induced by DBS in a Homogenous Brain.

Metabolic activity, due to baseline brain metabolism and increased metabolism in response to DBS, will act as a heat source inside the brain. Normally, blood perfusion regulates the brain temperature by convecting metabolic heat away. In this case—with metabolic heat—, the temperature of blood circulating in brain tissue was considered as 36.7° C.,²⁷ 0.3° C. lower than the initial brain temperature. In this case, how the interaction between metabolic heat generation and blood perfusion modulated DBS induced temperature increases was investigated. Prior to application of DBS, the various metabolic rates with blood perfusion rates were balanced so that baseline brain temperature remained at 37° C. The metabolic heat required to balance the initial brain temperature was calculated from Qₘ=C_{b}ρ_{b}tω_{b} (T−T_{b}), for blood perfusion values of 0.004, 0.008, and 0.012 ml/s/ml, a metabolic heat Qₘ of 4566, 9132, and 13698 W/m³, respectively, was applied. ²⁷Xiaojiang X, P Tikuisis, G Giesbrecht. A Mathematical Model for Human Brain Cooling During Cold-Water Near-Drowning. Journal of Applied Physiology 86:265-272, 1999.

Using these three initial settings—combination of balanced metabolic and perfusion rates—, the temperature increases—from 37° C.—due to DBS was studied. Temperature profiles were exactly the same as those in the study considering blood perfusion (at 37° C.) without metabolic activity. This was mathematically expected given the equality between metabolism and perfusion set above. As noted above, these temperature profiles were lower than those without metabolic heat and without blood perfusion. This can be explained by the increased blood flow capacity to both balance the metabolic heat and reduce Joule heat.

(d) The Case 3—Temperature Distribution Induced by DBS Considering the Inhomogeneous Physical Properties of the DBS Lead with and without a Tissue Encapsulation Layer.

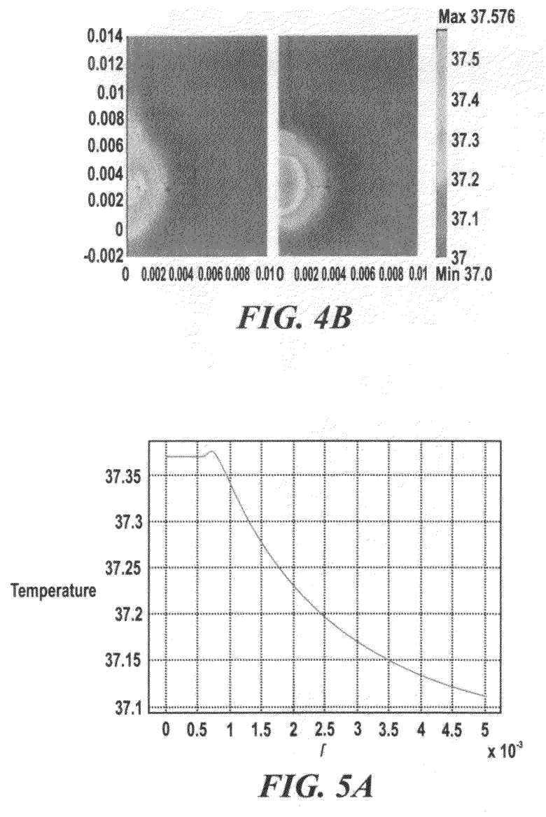

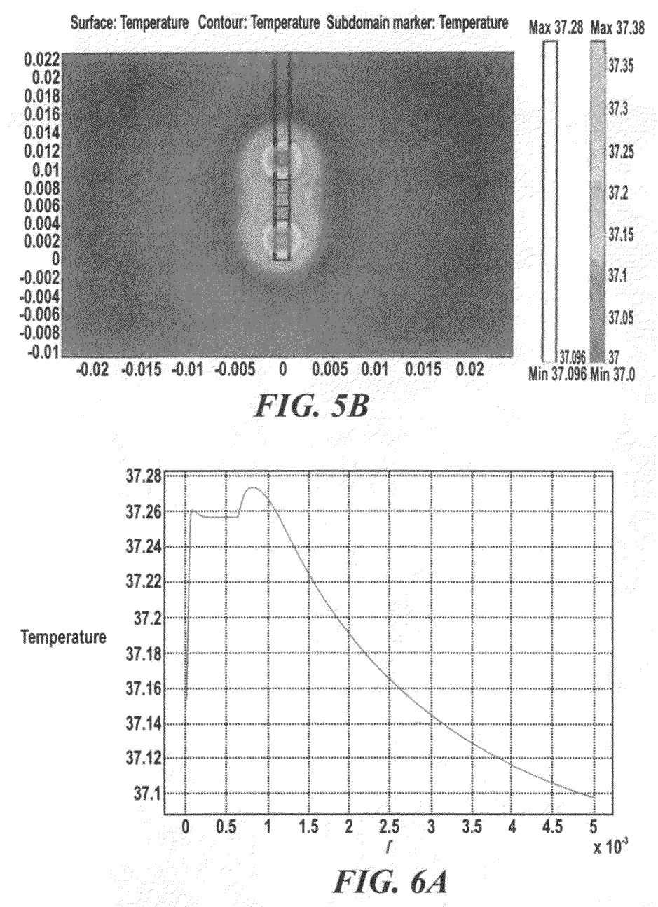

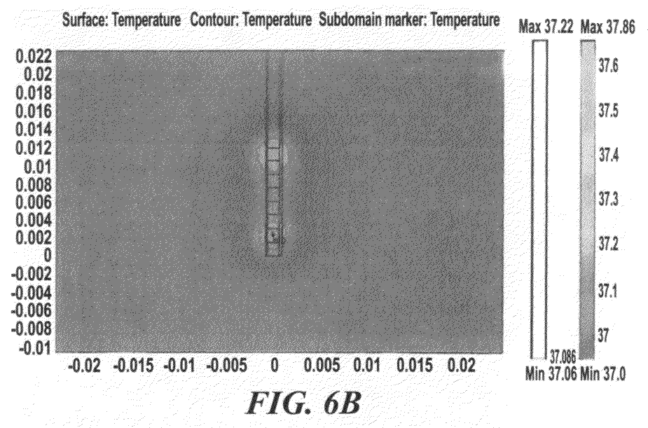

In this case, the thermal and electrical properties of the DBS leads were explicitly considered. Previously, the DBS leads were modeled as electrically and thermally insulated. The thermal conductivity was fixed, Kₑ=31 W/m° C., and electrical conductivity, σₑ₌4*10⁶ S/m of the DBS platinum/iridium electrodes. The thermal conductivity of the Medtronic DBS lead insulation material—Urethane—was considered present everywhere except the electrodes, as 0.026 W/m° C. In the simulations, a range of potential insulation material thermal conductivities (Kᵢ) from 0.026 W/m° C. to 20 W/m° C. was also considered in order to evaluate the effects of substitute insulation materials on DBS-induced temperature rises. FIG. 4A shows the temperature distribution over distance for different lead insulation thermal conductivity values (Kᵢ) using the high stimulation setting without blood perfusion and metabolic heat, and tissue thermal and electrical conductivity fixed at Kₜ=0.527 W/m° C. and σ=0.3 S/m, respectively. TABLE 2 shows that the peak tissue temperature decreased by 0.1-0.2° C. as a result of considering 19 lead properties. The insulation thermal conductivity (Kᵢ) acts as a heat-sink. FIGS. 4A and 4B illustrate how the insulation segments of the electrode could act as a heat-sink. The temperature was convected inside the lead insulation and reduced the heat from the tissue.

A sheath of encapsulation tissue around the DBS leads may form. The electrical 24 conductivity and width of the encapsulation tissue were previously estimated as σ_{dt}=0.15 S/m and 0.4 mm thick.²⁸ The thermal conductivity of the encapsulation tissue was treated as equal to that of the brain tissue, i.e. Kₜ=0.527 W/m° C. How this encapsulation tissue affected DBS-induced temperature increases was simulated. The tissue conditions were considered and included the properties of the DBS lead. Addition of an encapsulation layer in the model slightly reduced the peak temperature rise at the electrode surface—now inside the encapsulation layer—by 0.07-0.18° C. depending on the lead model and electrode configuration tested. ²⁸McIntyre C, S Mori, D Sherman, N Thakor, J Vitek. Electric Field and Simulating Influence Generated by Deep Brain Stimulation of the Subthalamic Nucleus. Clinical Neurophysiology; 115 (2004): 5 89-595.

### C. The Embodiments of the Present Invention

To mitigate DBS-induced temperature rises suggested by the simulations, the thermal conductivity of the insulating components of the leads should be increased.

The embodiments of the present invention include cooling the DBS leads and surrounding tissue by using passive/active heat-sinks. In one embodiment, portions of the DBS lead material is replaced with high thermal-conductivity material. The lead material then acts to carry away the heat from dangerous ‘hot spots.’ This method represents an effective and practical method for reducing tissue heating near DBS leads and would thus have tremendous clinical benefit. In another embodiment, the thermal conductivity of the insulating components of the DBS leads is changed. In yet another embodiment, the DBS leads are cooled by using heat-sinks. In yet another embodiment, the heat generated by different stimulation configurations is compared. In yet another embodiment, the dimensions of the device or device components are modified.

Different implantations of active heat-sink technologies may be used including those incorporating fluid flow. In an additional implementation of the heat-sink technology, the device may be modified—for example coated with a drug—that will induce the surrounding tissue to become more resistant to temperature increases—for example, by increasing tissue vasculature or changing tissue properties. In specific cases, existing devices and already implanted devices may be modified or retrofitted based on this heat-sink technology. This heat-sink technology would be effective during ‘normal’ device operation and during unexpected/faulty operation.

### D. The Impressions

It will be understood that each of the elements described above or two or more together may also find a useful application in other types of constructions differing from the types described above.

While the invention has been illustrated and described as embodied in a method to reduce heating at implantable medical devices including neuroprosthetic devices, however, it is not limited to the details shown, since it will be understood that various omissions, modifications, substitutions, and changes in the forms and details of the device illustrated and its operation can be made by those skilled in the art without departing in any way from the spirit of the present invention.

Without further analysis the foregoing will so fully reveal the gist of the present invention that others can by applying current knowledge readily adapt it for various applications without omitting features that from the standpoint of prior art fairly constitute characteristics of the generic or specific aspects of the invention.

TABLE 1

| | | | | PEAK | |
|---|---|---|---|---|---|
| | | | | TEMPERATURE | |
| | | | | (° C.) | |
| | ELECTRICAL | THERMAL | BLOOD | 3389 | 3387 |
| | CONDUCTIVITY | CONDUCTIVITY | PERFUSION | DBS | DBS |
| SECTION | σ (S/m) | Kₜ (W/m. ° C.) | ω_{b} (ml/s/ml) | LEAD | LEAD |
| I | 0.15 | 0.527 | 0 | 37.35 | 37.21 |
| | 0.20 | | | 37.47 | 37.28 |
| | 0.30 | | | 37.70 | 37.42 |
| | 0.35 | | | 37.82 | 37.48 |
| II | 0.30 | 0.45 | 0 | 37.82 | 37.48 |
| | | 0.50 | | 37.74 | 37.44 |
| | | 0.55 | | 37.67 | 37.40 |
| | | 0.60 | | 37.62 | 37.37 |
| III | 0.30 | 0.527 | 0 | 37.70 | 37.42 |
| | | | 0.004 | 37.61 | 37.34 |
| | | | 0.008 | 37.57 | 37.31 |
| | | | 0.012 | 37.54 | 37.29 |

TABLE 2

| | | | 3387 DBS LEAD | |
|---|---|---|---|---|
| | 3389 DBS LEAD | | Tₘₐₓ (° C.) | |
| | Tₘₐₓ (° C.) | | | ELEC- |
| Kᵢ | ELECTRODES | ELECTRODES | ELECTRODES | TRODES |
| (W/m · K) | 1 AND 4 | 1 AND 2 | 1 AND 4 | 1 AND 2 |
| 0.026 | 37.26 | 37.58 | 37.22 | 37.37 |
| 1 | 37.24 | 37.50 | 37.20 | 37.35 |
| 5 | 37.22 | 37.43 | 37.18 | 37.31 |
| 10 | 37.21 | 37.40 | 37.17 | 37.29 |

The invention claimed is:

1. A method to reduce heating or to change spatial distribution of heating at implantable medical devices including neuroprosthetic devices, comprising the step of increasing thermal conductivity of components of the implantable medical devices including the neuroprosthetic devices.

2. A method to reduce heating or to change spatial distribution of heating at implantable medical devices including neuroprosthetic devices, comprising the step of cooling the implantable medical devices including the neuroprosthetic devices by using heat-sinks.

3. A method to control heating or to change spatial distribution of heating at implantable medical devices including neuroprosthetic devices, comprising the step of replacing components of the implantable medical devices including the neuroprosthetic devices with specific thermal properties.

4. A method to reduce heating or to change spatial distribution of heating at implantable medical devices including neuroprosthetic devices, comprising the step of modifying/coating components of the implantable medical devices including the neuroprosthetic devices with a material or drug that will induce surrounding tissue to become more resistant to temperature increases.

5. A method to determine and reduce hazards relating to temperature increases at implantable medical devices including neuroprosthetic devices, comprising the step of using a modified bio-heat transfer model.

6. A method to control heating or to change spatial distribution of heating at implantable medical devices including neuroprosthetic devices, comprising the step of changing outer or inner surface dimensions and geometry of the device or device components.
